(19) United States
(12) Patent Application Publication
(10) Pub. No.: US 2004/0058327 A1
(43) Pub. Date: Mar. 25, 2004

(54) Method for using a blank matrix in a continuous format high throughput screening process

(76) Inventors: Jeffrey Y. Pan, Lake Forest, IL (US); Thomas A. Nemcek, Crystal Lake, IL (US); Carlos Gonzalez, Mundelein, IL (US); Eugene S. Maslana, Morton Grove, IL (US); Reza S. Sabet, Buffalo Grove, IL (US); Jennifer B. Donnelly, Wildwood, IL (US); David J. Burns, Lake Forest, IL (US); Duncan R. Groebe, Libertyville, IL (US); Usha Warrior, Green Oaks, IL (US)

Correspondence Address: STEVEN F. WEINSTOCK, ABBOTT LABORATORIES, 100 ABBOTT PARK ROAD, DEPT. 377/AP6A, ABBOTT PARK, IL, 60064-6008, US

(21) Appl. No.: 10/247,892

(22) Filed: Sep. 20, 2002

(51) Int. Cl. C12Q 1/68; C12M 1/34; G01N 33/543

(52) U.S. Cl. 435/6; 435/287.2; 436/518

(57) ABSTRACT

A method for testing a multiplicity of chemical entities for the ability of these chemical entities to enhance or inhibit a biological process. In one embodiment, the method comprises the steps of:

(a) providing a blank matrix having at least two major surfaces, the at least two major surfaces capable of receiving assay components and chemical entities;

(b) applying at least one chemical entity to at least one of the at least two major surfaces of the blank matrix, whereby an impregnated matrix is formed;

(c) applying to at least one of the at least two major surfaces of the impregnated matrix at least one assay component required for a biological process; and

(d) evaluating the ability of the at least one chemical entity to enhance or inhibit the biological process involving the at least one assay component.

In the preferred embodiments, a response indicative of an enhancement or an inhibition of the aforementioned biological process can be detected by a tracer, which can be introduced to the impregnated matrix as an assay component. The response so detected can be preserved in the form of an image of the at least one of the two major surfaces of the impregnated matrix.

### BACKGROUND OF THE INVENTION

1. Field of the Invention

This invention relates to a method for screening large numbers of chemical entities for a wide range of biological or biochemical activity, and more particularly, relates to the use of a blank matrix for such screening.

2. Discussion of the Art

A high throughput screening process is a process for testing large numbers of samples of chemical entities against a particular drug target in order to identify those chemical entities that could possibly elicit a desired response.

U.S. Pat. No. 5,976,813 describes an assay wherein a multiplicity of chemical entities is introduced into or onto a porous matrix that typically contains one or more assay components (herein referred to as Continuous Format High Throughput Screening or CF-HTS). A porous matrix suitable for the assay described in that patent can be prepared by adding, mixing, pouring, dispensing, or soaking these assay component(s) into the porous matrix. Porous matrices suitable for that assay can also be prepared by coupling, coating, binding, fixing, linking, conjugating or attaching assay components into or onto a surface of a matrix. Assay components include both biological reagents and chemical reagents.

The assay described in U.S. Pat. No. 5,976,813 provides many advantages over assays in which components are separated by impenetrable barriers into distinct wells. CF-HTS makes extraordinarily high-density screening of chemical entities routinely possible. Currently, 8,640 discrete chemical entities can be applied to a plastic sheet having the same footprint as a 96-well microtiter plate. In effect, miniaturization is achieved simply by limiting the volume of chemical entity available for the reaction in the porous matrix. By dispensing and drying chemical entities onto surfaces, such as plastic sheets, in discrete locations and highly packed arrays, and then transferring the chemical entities to a porous matrix, one can address all of the critical miniaturization issues associated with high-density (1536-well) microtiter plates. For example, maintaining dried chemical entities on surfaces for later reconstitution in a porous matrix eliminates concerns about loss of material due to evaporation during addition of assay components and chemical entities, and subsequent reaction times, as in the case with high-density microtiter plates.

Although it is possible that the desired effects observed in CF-HTS assays overlap, it is only necessary to retest the compounds that are located in proximity to the positions in the matrix where the desired effects are observed. Thus, it is possible to reduce 8,640 chemical entities to the 25 candidates in closest proximity to each of the observed effects and identify the desired chemical entities by retesting those 25 candidates nearest each observed effect (even with conventional 96-well microtiter plate technology). In addition, microfluidics is not required to dispense assay components in the CF-HTS format, because the assay components are dispensed and mixed in bulk. Only the chemical entities need to be dispensed by microfluidics and then dried for later use, depending upon the assay format.

Because the assay components are provided in the form of a homogeneous bulk solution for inclusion into a matrix, sample-to-sample differences due to variation in dispense volume is minimal. By comparison, the presence of wells in the conventional 96-well format typically results in significant sample-to-sample variations. Because only a single camera image of the reaction matrix is necessary, time and effort is saved in reading, comparing, and quantitating multiple wells. In addition to the benefits resulting from miniaturization (e.g., cost, throughput, usage of assay components, chemical entity usage), CF-HTS also provides surprising benefits such as the ability to handle most steps of the assay in bulk.

The assay described in U.S. Pat. No. 5,976,813 calls for uniformly distributing assay components throughout the porous matrix. Typically, the distribution of assay components is performed by bulk dissolution or suspension of the assay components within a flowing or malleable state of the matrix prior to solidification. Indeed, assay components that cannot diffuse readily through the matrix within a reasonable period of time must be incorporated in the porous matrix by means of bulk dissolution or bulk suspension. These assay components include, but are not limited to, high molecular weight proteins, cell membranes, and cells.

The introduction of assay components into a porous matrix by bulk dissolution or suspension can be a problem for four major reasons:

(1) the conditions needed to keep the material of the porous matrix in a flowing or malleable state may adversely affect some assay components; for example, the activity of some enzymes depends on the temperature; the higher temperatures needed to maintain some matrices in a flowing or malleable state might inactivate the enzymes required for the assay;

(2) the need to conserve rare and expensive assay components makes production of a large porous matrix inefficient because significant portions of the porous matrix may not be utilized in the final assay;

(3) the performance of the assay is dependent upon the thickness of the porous matrix; i.e., the thicker the porous matrix, the greater is the time needed for the assay components to mix, thereby increasing the length of time needed to complete a set of assays;

(4) the need to uniformly distribute insoluble assay components, such as cells, makes production of a large porous matrix difficult because the viscosity of the flowing or malleable material is high and because mixing of the matrix material as it solidifies is impractical.

Currently existing methods for adding assay components into the gel material of the porous matrix, such as, for example, contacting the matrix with a bulk solution, soaking, pouring, dipping, layering another gel onto the matrix, layering another substrate onto the matrix, can result in significant changes in the distribution of any substances that are capable of diffusing freely out of the porous matrix. For example, chemical entities will diffuse out of the matrix and into the bulk solution if the entire matrix is soaked for an extended period of time in order to introduce another assay component into the matrix.

### SUMMARY OF THE INVENTION

This invention provides a method for testing a multiplicity of chemical entities for the ability of these chemical entities to enhance or inhibit a biological process. In one embodiment, the method comprises the steps of:

(a) providing a blank matrix having at least two major surfaces, the at least two major surfaces capable of receiving assay components and chemical entities;

(b) applying at least one chemical entity to at least one of the at least two major surfaces of the blank matrix, whereby an impregnated matrix is formed;

(c) applying to at least one of the at least two major surfaces of the impregnated matrix at least one assay component required for a biological process; and

(d) evaluating the ability of the at least one chemical entity to enhance or inhibit the biological process involving the at least one assay component.

One or more additional chemical entities or one or more additional assay components can be applied to at least one of the at least two major surfaces of the impregnated matrix. In the preferred embodiments, a response indicative of an enhancement or an inhibition of the aforementioned biological process can be detected by a tracer, which can be introduced to the impregnated matrix as an assay component. The response so detected can be preserved in the form of an image of the at least one of the two major surfaces of the impregnated matrix.

A variation of the foregoing embodiment comprises the steps of:

(a) providing a blank matrix having at least two major surfaces, the at least two major surfaces capable of receiving assay components and chemical entities;

(b) applying at least one assay component required for a biological process to at least one of the at least two major surfaces of the blank matrix, whereby an impregnated matrix is formed;

(c) applying to at least one of the at least two major surfaces of the impregnated matrix at least one chemical entity; and

(d) evaluating the ability of the at least one chemical entity to enhance or inhibit the biological process involving the at least one assay component.

One or more additional chemical entities or one or more additional assay components can be applied to at least one of the at least two major surfaces of the impregnated matrix. As in the previous embodiment, a response indicative of an enhancement or an inhibition of the aforementioned biological process can be detected by a tracer, which can be introduced to the blank matrix or to the impregnated matrix as an assay component. As in the previous embodiment, the response so detected can be preserved in the form of an image of the at least one of the two major surfaces of the impregnated matrix.

A blank matrix can be a porous or non-porous matrix. The blank matrix is preferably a porous matrix. The blank matrix is capable of accommodating a biological reaction or a chemical reaction. The blank matrix lacks (1) any chemical entity that is being tested for a response involving the enhancement or the inhibition of a given biological process and (2) any assay component that is being used in the biological process for the specific purpose of generating a detectable response in the presence of the chemical entity.

The blank matrix is preferably formed from an agarose gel, filter paper, or blotting paper. The chemical entity is preferably a molecule having low molecular weight, a peptide, or an antibody. An assay component designated as a biological reagent is preferably an enzyme, a substrate for an enzyme, or a cell. An assay component designated as a chemical reagent is preferably a low molecular weight organic compound or an inorganic compound. Although the chemical entities can be applied to a major surface of a blank matrix or an impregnated matrix by any of several methods, the preferred methods include, but are not limited to, spraying, drop-wise addition, pin transfer, transfer from a transfer surface to a matrix, such as, for example, matrix-to-matrix transfer, paper-to-matrix transfer, and reconstitution from dried or frozen spots. Although the assay components can be applied to a major surface of a blank matrix or an impregnated matrix by any of several methods, the preferred methods include, but are not limited to, transfer from a transfer surface to a matrix, such as, for example, matrix-to-matrix transfer, paper-to-matrix transfer, spraying, pin transfer, and reconstitution from dried or frozen spots. Chemical entities and assay components can also be applied to a blank matrix or to an impregnated matrix by pouring or by dipping the matrix into a liquid for short periods of time. After being dispensed, assay components and chemical entities may be able to diffuse into the core of the matrix, i.e., below the major surfaces of the matrix, within a reasonable period of time, thereby making biological or chemical activity and the successful detection thereof possible. It is preferred that the at least one chemical entity be applied to a major surface of the blank matrix before the at least one assay component is applied to a major surface of the impregnated matrix. However, it is within the scope of the method of this invention to apply the at least one assay component to a major surface of the blank matrix before the at least one chemical entity is applied to a major surface of the impregnated matrix.

The method of this invention provides numerous advantages relative to previously known methods of performing continuous format high throughput screening. The advantages include the following:

(a) reduction in consumption of assay components;

(b) increased throughput;

(c) simplified production of the matrix;

(d) capability of carrying out a multiplicity of assays involving a plurality of chemical entities at a plurality of concentrations and a plurality of assay components at a plurality of concentrations in the same matrix; and

(e) capability of enhanced automation.

### BRIEF DESCRIPTION OF THE DRAWINGS

FIGS. 1A, 1B, 1C, 1D, 1E, 1F, and 1G make up a set of schematic diagrams that illustrate one embodiment of the method of this invention.

### DETAILED DESCRIPTION

As used herein, the expression “chemical entity” means any biological or chemical substance of known or unknown organic or inorganic composition having known or unknown biological and chemical effect. The purpose of the screening procedure described herein is to determine the effect of a given chemical entity on a biological process. The expression “assay component” means a chemical reagent or a biological reagent involved in an assay to test for a response involving the enhancement or the inhibition of a given biological process. The expression “biological reagent” means a reactive material derived from a biological source, which material is involved in a biological process that is capable of generating a detectable signal in an assay. The biological reagent can be modified or unmodified after derivation from its source. Representative examples of biological reagents include nucleic acids, proteins, and other synthetic or natural macromolecules; cells; cell lysates; cell membranes; biological extracts; organelles; and other complex biological entities and mixtures; and small molecules such as, for example, inhibitors, substrates, peptides, dyes, nucleotides, cofactors. The expression “chemical reagent” means a reactive material derived from a chemical source, which material is involved in a biological process that is capable of generating a detectable signal in an assay. The chemical reagent can be modified or unmodified after derivation from its source. Representative examples of chemical reagents include polymers, organic molecules, and inorganic molecules. The expression “blank matrix” means a matrix lacking (1) any chemical entity that is being tested for a response involving the enhancement or the inhibition of a given biological process and (2) any assay component that is being used in the biological process for the specific purpose of generating a detectable response in the presence of the chemical entity. The expression “blank matrix”, that is, the definition of “blank matrix”, is not intended to exclude those assay components, such as, for example, water, buffers, and salts, that are present merely to provide a suitable environment for performing the test for a response involving the enhancement or the inhibition of a given biological process. The expression “impregnated matrix” means a blank matrix to which at least one of a chemical entity or an assay component has been introduced by one of the steps of the method of this invention. The term “response” means a detectable result that indicates an interaction involving at least one chemical entity and at least one assay component.

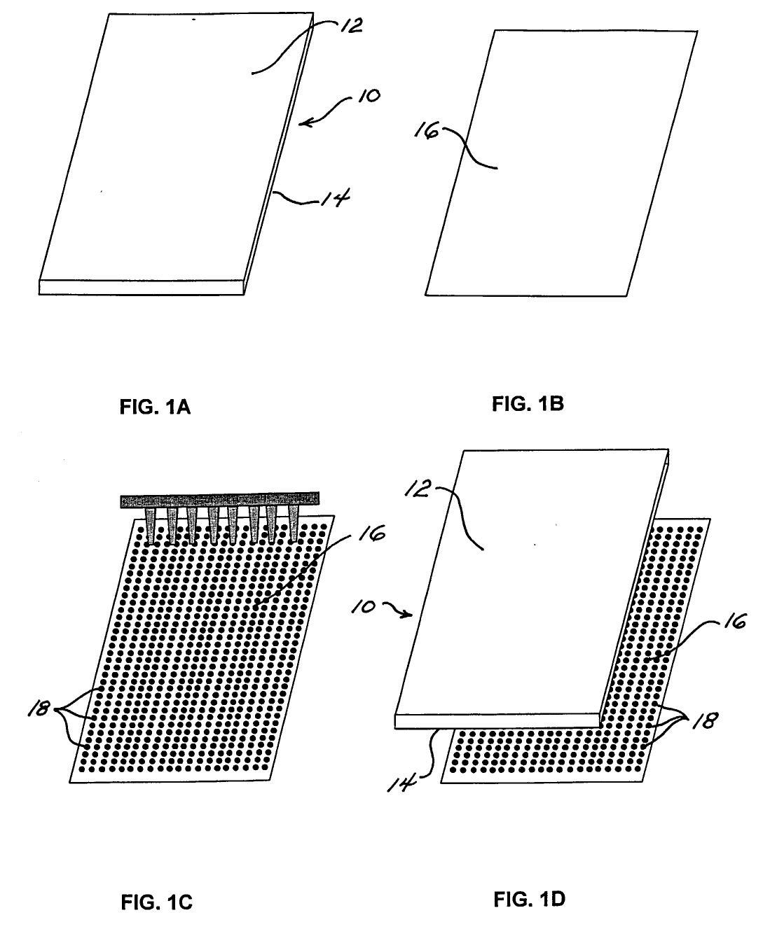

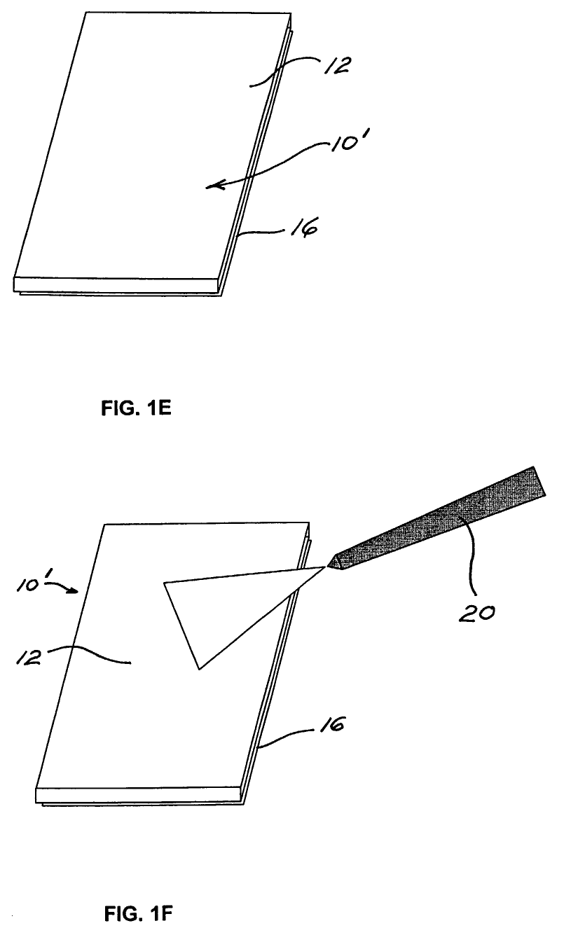

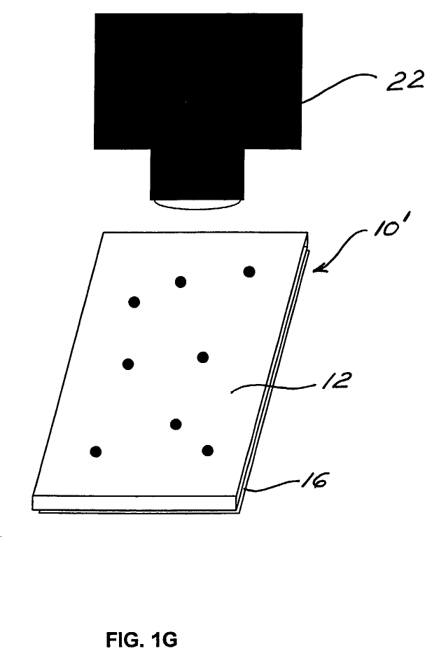

Referring now to FIGS. 1A, 1B, 1C, 1D, 1E, 1F, and 1G, the embodiment in which chemical entities are introduced to a blank matrix will be described. It should be noted that the embodiment in which assay components are introduced to a blank matrix is substantially similar, with the primary exception being in the nature of the material being introduced to the blank matrix. Referring now to FIG. 1A, a blank matrix 10 suitable for use in the process of this invention is shown. The blank matrix 10 has a first major surface 12 and a second major surface 14. FIG. 1B shows a transfer surface 16 suitable for applying chemical entities to at least one major surface of the blank matrix 10. FIG. 1C shows chemical entities 18 being applied to the transfer surface 16 of FIG. 1B. In order to apply the chemical entities to a major surface of the blank matrix 10, the transfer surface 16 is positioned such that the chemical entities 18 thereon are transferred by diffusion from the transfer surface 16 to at least one major surface 14 of the blank matrix 10. FIG. 1D shows the major surface 14 of the blank matrix 10 being brought into contact with the transfer surface 16 that bears chemical entities 18. After the chemical entities are transferred to the blank matrix 10, the blank matrix 10 that has been impregnated with the chemical entities 18 is referred to as the impregnated matrix 10′. In one embodiment of the method of this invention, the transfer surface 16 is separated from the impregnated matrix 10′. In another embodiment, as shown in FIG. 1E, it is not necessary to separate the transfer surface 16 from the major surface 14 of the impregnated matrix 10′ for subsequent steps in the assay. It should be noted that although it is preferable to apply chemical entities to a major surface of the blank matrix 10 by means of a transfer surface 16, chemical entities 18 can be applied to a major surface of the blank matrix 10 by other methods, such as, for example, transferring by pipettes, spraying. If the blank matrix 10 is sufficiently thin, such as, for example, having a thickness of less than about 1 mm, chemical entities, or assay components, can be applied to either the major surface 12 or the major surface 14 of the blank matrix 10. FIG. 1F shows a dispensing device 20, such as, for example, a sprayer, suitable for applying one or more assay components to a major surface of the impregnated matrix 10′. FIG. 1F also shows the impregnated matrix 10′ after the one or more assay components have been applied to the major surface 12 thereof. FIG. 1G shows equipment 22 that is suitable for preparing an image of the major surface 12 of the impregnated matrix 10′ after sufficient time for biological and chemical reactions to occur has elapsed, whereby the extent of the effects of the chemical entities can be determined. Such equipment may include, but is not limited to, imaging, reading, scanning, or detecting equipment.

The method of this invention can employ a wide range of materials for preparing blank matrices. Materials that are suitable for preparing blank matrices that are suitable for use in this invention include, but are not limited to, gels, preferably hydrogels, such as, for example, agarose, polyacrylamide, or the like, membranous materials, filtering materials, such as, for example, filter paper, paper materials, such as, for example, blotting paper, paper fibers, and polymeric materials. Polymeric materials that are suitable for preparing blank matrices include, but are not limited to, natural, synthetic, semi-synthetic polymeric materials. Representative examples of the forgoing materials, include, but are not limited to, proteins, carbohydrates, polystyrene, polypropylene, polycarbonate, polyester, polyvinylidene chloride, and polyethylene. Other materials from which blank matrices can be formed include, but are not limited to, fibrous materials, such as, for example, paper fiber, glass fiber. Mineral based substances, such as, for example, silica, can also be used to prepare blank matrices.

A blank matrix can be prepared by pouring and casting a material capable of forming a gel, such as, for example, agarose, into a mold. A blank matrix can be prepared by molding, such as, for example, extrusion molding. The material for preparing a blank matrix can be flowing or non-flowing. The material that is poured and cast into the mold is transformed into a blank matrix by a change in one or more of environmental, biological, or chemical conditions, such as, for example, changes in temperature, pH, or exposure to radiation, including exposure to light. A blank matrix can be prepared by dispersing, suspending, or dissolving a polymeric material in a liquid medium, such as an aqueous medium, and changing one or more of environmental, biological, or chemical conditions such that the liquid phase becomes non-flowing, i.e., becomes a gel, within a specified period of time. Alternatively, blank matrices that are suitable for use in this invention are commercially available from vendors supplying fibrous materials, such as, for example, filter paper, polymeric materials, protein-based materials, and mineral-based materials. Blank matrices can be obtained from such manufacturers as Bio-Rad Laboratories, Inc. (Hercules, Calif.), The Perkin Elmer Corporation, Life Sciences (Boston, Mass.), and Promega Corporation (Madison, Wis.).

A matrix, preferably a non-porous matrix, can be used as a transfer surface to spatially fix chemical entities, biological reagents, or chemical reagents, or any combination of the foregoing onto a blank matrix or an impregnated matrix. A transfer surface can be prepared by coupling, coating, binding, fixing, linking, conjugating, or attaching assay components or chemical entities onto a surface of a matrix, preferably a non-porous matrix. The use of a matrix for this purpose in this invention fixes the position of one or more of the assay components. Polymeric materials that are suitable for preparing a transfer surface by means of a non-porous matrix include, but are not limited to, polystyrene, polypropylene, polycarbonate, polyester, polyvinylidene chloride, and polyethylene. Other materials from which a non-porous matrix can be formed include, but are not limited to, paper, fibrous materials, such as, for example, paper fiber, glass fiber, and mineral-based materials, such as silica.

The transfer surface is preferably a major surface of a sheet of polymeric material. It is preferred that the chemical entities neither mix nor overlap on the transfer surface, and that each chemical entity be in a specified location on the transfer surface. However, it is also within the scope of this invention that chemical entities can be applied randomly to a transfer surface, in which case those chemical entities that provide a response indicative of an enhancement or an inhibition of a given biological process can be identified by techniques other than specifying the initial location thereof. Such techniques include, for example, the use of encoded combinatorial chemical libraries. In an encoded combinatorial chemical library, each chemical entity may be synthesized to include a unique tag, the presence of which can be detected by physical means (e.g., NMR, mass spectroscopy) or chemical means (fluorometric, radiometric). The subsequent identification of the unique tag identifies the chemical entity of which it is a part. Thus, it is not necessary to know the initial position of the chemical entity on the transfer surface if the chemical entity can be identified by means of the identity of its tag. When the transfer surface is placed in face-to-face contact with the major surface of a blank matrix, the chemical entities dissolve and diffuse into the blank matrix, preferably porous matrix, in locations corresponding to their specified locations in the initial array on the transfer surface. A transfer surface can also be used to introduce assay components to a blank matrix.

When chemical entities and assay components are introduced into the blank matrix, or the impregnated matrix, the chemical entities and assay components can be attached to the blank matrix, or the impregnated matrix, by covalent or non-covalent binding, by specific or nonspecific binding interactions with the matrix. The blank matrix, or the impregnated matrix, can be non-derivatized, derivatized, or otherwise pre-treated to facilitate the attachment of the chemical entities and assay components thereto. After attachment to the blank matrix or to the impregnated matrix, the chemical entity or the assay component is spatially fixed, whereby diffusion of the chemical entity or the assay component is restricted for the purposes of the assay. It should be noted that either the chemical entities must be able to diffuse to the assay components attached to the matrix or the assay components must be able to diffuse to the chemical entities attached to the matrix.

Assay components (i.e., biological reagents and chemical reagents) that are suitable for use in this invention include, but are not limited to, macromolecules, such as, for example, nucleic acids, proteins, and other synthetic or natural macromolecules; cells; cell lysates; biological extracts; organelles and other complex biological entities and mixtures; and small molecules, such as, for example, salts, inhibitors, substrates, peptides, dyes, nucleotides, cofactors, ions, and solvents.

### Operation

In the preferred embodiments of this invention, chemical entities are dispensed onto a transfer surface in a highly packed array and in discrete locations and allowed to dry prior to application via transfer to a blank matrix, preferably a blank porous matrix. In a typical application, the transfer surface containing the chemical entities is positioned in face-to-face contact with a major surface of the blank matrix such that all of the chemical entities are transferred by diffusion from the transfer surface to the major surface of the blank matrix. As stated previously, the blank matrix to which chemical entities or assay components or both chemical entities and assay components have been applied is referred to as an impregnated matrix. Additional matrices containing assay components required for the high throughput screening operation can then be brought into face-to-face contact with the impregnated matrix. The chemical entities and assay components diffuse and interact within the impregnated matrix. The effect of the chemical entities on the interaction among the assay components in the impregnated matrix can be determined both qualitatively and quantitatively by measurement of colorimetric tracers, radiometric tracers, fluorometric tracers, or combinations of the foregoing, which are typically included as assay components. For example, the effect of a given chemical entity on the reaction between an enzyme and a substrate for the enzyme or on the interaction between a ligand and a receptor for the ligand, can be determined by means of the foregoing tracers. The signals resulting from the tracers, or equivalents thereof, can be preserved by means of equipment for imaging, reading, scanning, detecting or the like. Such equipment includes, but is not, limited to gel documentation and imaging systems, spectrophotometric scanners, CCD cameras, film, phosphorimagers, and scintillation detection devices.

In an alternative embodiment, chemical entities can be directly applied onto a major surface of a blank matrix, or an impregnated matrix, in an array, by microfluidics, which can involve dispensing the chemical entities directly onto the major surface of the blank matrix, or the impregnated matrix, by, for example, spraying, drop-wise addition, pipette transfer, pin transfer, bead transfer, reconstitution from frozen or dried spots, or contacting the surface of the matrix with a liquid, wherein the volume of each chemical entity dispensed is low enough so that the chemical entities do not substantially overlap within the matrix.

A preferred method for introducing chemical entities at a plurality of concentrations onto a major surface of a blank matrix, or an impregnated matrix, involves the use of pipettes capable of dispensing fluids in small amounts or the use of a pin transfer tool. In certain situations, chemical entities can be introduced onto a major surface of a blank matrix, or an impregnated matrix, in the form of a solid.

The impregnated matrix to which the chemical entities have been applied does not have certain critical assay components contained on or within the matrix. These particular critical assay components can be applied to a major surface of the impregnated matrix by any of several methods, including, but not limited to, pouring, spraying, transferring by surface-to-surface contact, or soaking. These critical assay components include those components that are involved in a biological process for which is sought a response relating to enhancement or inhibition of the biological process by a chemical entity, such as, for example, cells, enzymes, substrates for enzymes; however, these critical assay components exclude those assay components that are present merely to provide a suitable environment for performing the test for the response involving the enhancement or the inhibition of the given biological process, such as, for example, water, buffers, and salts. In certain situations, assay components can be introduced onto a major surface of an impregnated matrix, in the form of a solid.

It is possible to introduce such chemical entities as combinatorial compounds attached to a bead into a blank matrix or impregnated matrix by dispensing such beads containing these compounds randomly or in an ordered array onto a transfer surface, such as the surface of a polymeric sheet or a filter material. The beads can then be treated to release (cleave) the compounds if they are covalently attached to the beads by a labile linker by means of photocleavage or gas-phase acid cleavage, which methods are well known in the art. Each compound is then non-covalently associated with the area originally occupied by the bead to which it was attached, and the dry compounds can then be introduced into or onto a blank matrix or impregnated matrix by contacting a major surface of the matrix with the polymeric sheet or filter material carrying the beads. The beads may be left in contact with the blank matrix or impregnated matrix for the remainder of the assay, as in the situation where the beads are dispensed onto a polymeric sheet. Alternatively, the beads may be removed from the blank matrix or impregnated matrix after a period of time sufficient for chemical entities to diffuse into the matrix merely by removing the polymeric sheet from the matrix.

An alternative method for introducing chemical entities such as discrete compounds into a blank matrix or impregnated matrix involves adhering or otherwise non-covalently attaching each compound into or onto beads, and then dispensing the beads randomly or in an ordered array onto a major surface of a polymeric sheet or filter in such a way that the chemical entities cannot move from one bead to another. Then, the surface bearing the beads can be contacted with a major surface of the matrix to dispense the chemical entities. This procedure completely eliminates the need for handling liquids that are present in small volumes.

An alternative method for dispensing chemical entities or assay components onto a major surface of an impregnated matrix, in an array, is to dispense chemical entities or assay components onto a second matrix, preferably a porous matrix, such as a filter, where the volume of each chemical entity or assay component dispensed is sufficiently low that the chemical entities or assay components so dispensed do not overlap within the second matrix. Upon surface-to-surface contact of the second matrix with the impregnated matrix, which has a higher liquid content than the second matrix, the chemical entities or assay components diffuse to initiate the assay.

When chemical entities introduced onto a major surface of a blank matrix or impregnated matrix are introduced at a high density, diffusion of the chemical entities is likely to occur in the matrix in such a way that any positive response resulting from the interaction of one or more chemical entities with the assay components may overlap the initial location (i.e., the location prior to diffusion) of one or more other chemical entities introduced into the blank matrix or the impregnated matrix. Consequently, there may be more than one candidate chemical entity within an area of positive response, as multiple chemical entities will be initially present in a zone of activity. Chemical entities may diffuse together during the period of the assay, but each chemical entity will have its own symmetrical spatial gradient and will not be quantitatively mixed at any one location. Therefore, the center of the zone of activity of a given chemical entity can still be correlated to the precise initial location of the given chemical entity. In practice, responses are sufficiently rare that retesting a multiplicity of chemical entities to ensure the identification of active chemical entities for each zone of activity is trivial. As stated previously, it is within the scope of this invention that chemical entities, such as, for example, encoded combinatorial chemical libraries, can be applied randomly to a transfer surface, in which case those chemical entities that provide a response indicative of an enhancement or an inhibition of a given biological process can be identified by a technique other than specifying the initial location thereof.

An alternative embodiment of the invention involves introducing physical barriers into the blank matrix to limit the distance that chemical entities can diffuse. This format is, in effect, partially non-continuous. For example, a blank matrix, preferably a porous matrix, can be molded around a fine mesh or screen such that the blank matrix is divided into numerous discrete and independent regions. Chemical entities and assay components can then be applied to the discrete and independent regions of the divided matrix. Alternatively, an impregnated matrix, preferably a porous matrix, can be divided into numerous discrete and independent regions by inserting a fine screen into the matrix to isolate individual portions of the matrix. If necessary, chemical entities and assay components can then be applied to the discrete and independent regions of the divided matrix. Because of the division process, in each discrete and independent region the assay is completely independent of other assays in the matrix. Furthermore, there is no diffusion of chemical entities or assay components from one discrete and independent region to another. This embodiment eliminates some of the advantages of continuous format high throughput screening (1) by introducing statistically significant deviations between assays and (2) by fixing the volume, thereby limiting the signal for high-density arrays. However, this embodiment still retains some of the advantages of continuous format high throughput screening for those assay components dispensed in a continuous fashion. Moreover, mixing of chemical entities within the matrix is eliminated.

It should be noted that one or more assay components can be applied to a blank matrix to form an impregnated matrix, and one or more chemical entities can then be applied to the impregnated matrix. The methods of introduction can be same as those employed to introduce assay components to an impregnated matrix and chemical entities to a blank matrix. The impregnated matrix to which the chemical entities and the assay components have thus been introduced can be observed, imaged, and analyzed in the same manner that the impregnated matrix in which introduction of chemical entities precedes introduction of assay components can be observed, imaged, and analyzed.

There are numerous advantages brought about by the method of this invention. The method of this invention results in reduction in consumption of assay components. In contrast to those assays in which assay components are cast into a matrix, assay components can be sprayed onto the surface of a blank or impregnated matrix. The elimination of the second matrix, in effect, increases the concentration of assay components in the single matrix. By using a spray to apply assay components to the matrix, the distance needed for diffusion is reduced, thereby increasing reaction kinetics, with ultimate result being higher throughput. When assay components are cast into a matrix, the elevated temperatures required to maintain the matrix in the liquid state are detrimental to the stability of some assay components. In addition, the uniform distribution of assay components, such as, for example, cells, may be difficult on account of the viscosity of the molten material of the matrix. A multiplicity of assays involving (a) different chemical entities or (b) different concentrations of a chemical entity or (c) different concentrations of different chemical entities can performed in a single matrix. Likewise, a multiplicity of assays involving (a) different assay components or (b) different concentrations of an assay component or (c) different concentrations of different assay components can performed in a single matrix. For example, in a typical matrix, (a) 96 different chemical entities can be tested or (b) 96 concentrations of the same chemical entity can be tested or (c) 24 different concentrations of four (4) different chemical entities can be tested. Likewise, eight (8) different concentrations of four (4) different chemical entities can be tested in three (3) different arrangements of assay components. The method of this invention can be readily automated because preformed blank matrices are highly reproducible and highly uniform. Moreover, optimization of assays is simplified because a multiplicity of arrangements of assay components can be evaluated simultaneously to determine the concentrations of assay components that provide the best signal. Furthermore, blank matrices can be purchased or prepared and then stored far in advance of an assay to be performed. Because the blank matrices lack assay components that may have relatively brief shelf lives, the likelihood that the matrices will be unsuitable for use after long-term storage is greatly reduced. In addition, matrices preserved in storage are immediately available for use in any assay that requires them.

The following non-limiting examples further illustrate this invention.

### EXAMPLES

### Example 1

A blank matrix having the approximate dimensions 127 mm×100 mm×0.75 mm is provided. For this example, the blank matrix comprises 1% agarose in water. A blank matrix having similar features can be obtained commercially from Bio-Rad Laboratories, Inc. The blank matrix is equilibrated in an aqueous buffer necessary for the assay by soaking the matrix in a quantity of the aqueous buffer. This aqueous buffer comprises:

25 mM Tris, pH 8.0

137 mM NaCl

2.7 mM KCl

1 mM MgCl₂

2% glycerol

The matrix is removed from the buffer and allowed to dry for 10 minutes at room temperature. A sheet for transferring a chemical entity is provided. This sheet typically consists of a thin sheet (0.5 mm) of polystyrene having a major surface dimension of approximately 8.5 cm by 12.5 cm. This sheet can contain 96 or more chemical entities spotted at least 1 mm apart over the major surface of the sheet. One major surface of the blank matrix is placed in face-to-face contact with the major surface of the sheet for transferring the chemical entity. The thus contacted matrix is incubated at room temperature for 10 minutes. A preparation of enzyme (histone deacetylase, 80 μl nuclear cell extract in 2 ml of the aqueous buffer described above) is applied to a major surface of the impregnated matrix by means of spraying, and the impregnated matrix is then incubated for 10 minutes at room temperature. A preparation of substrate (100 μM in 2 ml of the aqueous buffer described above) is applied to a major surface of the impregnated matrix by means of spraying, and the impregnated matrix is then incubated for 30 minutes at room temperature. A preparation of reaction developer (1:50 dilution of stock solution in the aqueous buffer previously described) is applied to a major surface of the impregnated matrix by means of spraying, and the impregnated matrix is then incubated for 5 to 10 minutes at ambient temperature. Substrate and developer for the reaction can be a proprietary, commercially available product (fluorescent lysine having the trademark Fluor de Lys) from BioMol Research Labs, Inc (Plymouth Meeting, Pa.). The impregnated matrix is imaged by means of an Eagle Eye II imaging system (excitation wavelength is 360 nm; emission wavelength is 460 nm).

The parameters of the spraying step of the method can be established empirically and depend upon the required conditions of the particular assay. In this example, the air pressure required to produce a spray from a commercially available model airbrush is maintained at approximately 5 psi. The volume of the solution sprayed (2 ml) can be applied to the impregnated matrix within 30 seconds. The distance from the impregnated matrix and the angle of application can also be determined empirically to avoid tearing or lifting of the matrix by the spray pressure and to avoid uneven application of the assay components to the matrix. A flat spray pattern distributes the liquid as a flat- or sheet-type spray. The flat spray pattern is formed by use of an elliptical orifice, or by a round orifice tangential to a deflector surface. In the elliptical orifice design, the axis of the spray pattern is a continuation of the axis of the inlet pipe connection. In the deflector design, the deflection surface diverts the spray pattern away from the axis of the inlet pipe connection. In the deflector design, the deflection surface diverts the spray pattern away from the axis of the inlet pipe connection. Straight-through elliptical orifice spray nozzles normally produce flat spray patterns with tapering edges. This characteristic is useful in establishing overlapping patterns between adjacent sprays on a multiple-nozzle spray head. For example, in the case of a sprayer having four identical spray nozzles, if the density of the spray from each nozzle is equal from one end of the spray pattern to the other, and if the spray patterns from each nozzle were to overlap, then the regions between adjacent spray patterns from adjacent nozzles would contain twice as much sprayed material as would the central region of each spray pattern. However, if the spray pattern from each nozzle were “tapered” such that the spray density, not the spray area, diminished toward either end of the pattern, then one could separate adjacent spray nozzles at a distance such that the overlapping regions between the adjacent spray patterns would have the same amount of sprayed material as the central region of each spray pattern. Thus, the resulting distribution across the entire sprayed surface can therefore be uniform.

### Example 2

The purpose of this example is to establish the feasibility of performing one or more kinase assays by means of a blank matrix.

In this example, the assay components comprise radioactively tagged adenosine triphosphate (ATP), the kinase enzymes of interest, and the substrates for the kinase enzymes of interest. The substrates are affinity tagged with biotin. In this example, a membrane that is coated with streptavidin is also employed. In an uninhibited reaction, the enzyme performs the task of phosphorylation, that is, acting as a catalyst, cleaves the radioactive phosphate from the ATP molecule and attaches the radioactive phosphate to the biotinylated substrate. The chemical entities to be tested will either inhibit the enzyme to some degree or have no effect.

Streptavidin is a tetrameric protein that binds very strongly to the small molecule biotin. This strong bond gives the membrane the ability to capture the substrate, thereby allowing the level of radioactivity to be measured. The amount of radioactive signal can be correlated to the effects of the various chemical entities on the ability of the enzyme to perform its task of phosphorylation.

A blank matrix having the approximate dimensions 127 mm×100 mm×0.5 mm is provided. This matrix is placed over a single layer streptavidin-coated membrane. The resulting matrix/membrane complex is then placed on the deck of a Cartesian synQuad™ system for dispensing materials for use in an assay. The system is manufactured by Cartesian Technologies, Incorporated, Irvine, Calif. Chemical entities in various concentrations are prepared and placed in a 96-well plate. Enzymes in various concentrations are prepared and placed in a 96-well plate. Solutions containing radioactively tagged ATP and substrates for the enzymes in various concentrations are prepared and placed in a 96-well plate. Each of these 96-well plates was placed on the deck of the Cartesian synQuad™ system for dispensing. The Cartesian synQuad™ system aspirates the chemical entities from the 96-well plate and dispenses them in droplets directly onto the surface of the blank matrix in a specified array. After the chemical entities have diffused into the blank matrix, which requires approximately 5 minutes, the enzymes are then aspirated and dispensed directly onto the surface of the impregnated matrix in a specified array. After little or no delay, the solutions of ATP and the substrates are then aspirated and dispensed directly onto the surface of the impregnated matrix in a specified array. After all the assay components have been dispensed, the impregnated matrix is removed from the deck and covered to prevent it from drying out during a 2-hour incubation period. After the incubation period has elapsed, the material of the matrix is washed off the streptavidin-coated membrane, and the resulting washed membrane placed into a PhosphoImager for development of the resulting images, if any.

Typical parameters for the foregoing process include, but are not limited to, the following:

(1) Five concentrations of inhibitor (the chemical entity) can be tested: 1.8 mM, 3 mM, 4.5 mM, 6 mM, and 9 mM. No inhibitor (a control) is also tested.

(2) Each droplet of liquid dispensed (chemical entities and assay components) typically comprises from about 25 to about 35 nanoliters in volume and the distance between the centers of each droplet on the surface of the matrix is from about 2.5 to about 3.0 mm.

Various modifications and alterations of this invention will become apparent to those skilled in the art without departing from the scope and spirit of this invention, and it should be understood that this invention is not to be unduly limited to the illustrative embodiments set forth herein.

What is claimed is:

1. A method for testing at least one chemical entity for the ability of said chemical entity to enhance or inhibit a biological process, said method comprising the steps of:
(a) providing a blank matrix having at least two major surfaces, said at least two major surfaces capable of receiving assay components and chemical entities;
(b) applying at least one chemical entity to at least one of said at least two major surfaces of said blank matrix, whereby an impregnated matrix is formed;
(c) applying to at least one of said at least two major surfaces of said impregnated matrix at least one assay component required for a biological process; and
(d) evaluating the ability of said at least one chemical entity to enhance or inhibit said biological process involving said at least one assay component.

2. The method of claim 1, wherein step (d) includes the step of detecting a response that indicates whether said at least one chemical entity enhances or inhibits said biological process involving said at least one assay component.

3. The method of claim 2, further including the step of preparing an image of said detected response.

4. The method of claim 1, wherein said at least one assay component is applied to at least one of said at least two major surfaces of said impregnated matrix by means of a transfer sheet.

5. The method of claim 1, wherein said at least one assay component is applied to at least one of said at least two major surfaces of said impregnated matrix by means of liquid dispensing apparatus.

6. The method of claim 1, wherein said at least one assay component is applied to at least one of said at least two major surfaces of said impregnated matrix in the form of a solid.

7. The method of claim 1, wherein said at least one chemical entity is applied to at least one of said at least two major surfaces of said blank matrix by means of a transfer sheet.

8. The method of claim 1, wherein said at least one chemical entity is applied to at least one of said at least two major surfaces of said blank matrix by means of liquid dispensing apparatus.

9. The method of claim 1, wherein said at least one chemical entity is applied to at least one of said at least two major surfaces of said blank matrix in the form of a solid.

10. The method of claim 1, wherein at least two different assay components are applied to said impregnated matrix.

11. The method of claim 1, wherein at least two different chemical entities are applied to said blank matrix.

12. The method of claim 1, wherein one or more additional chemical entities or one or more additional assay components can be applied to at least one of said at least two major surfaces of said impregnated matrix.

13. The method of claim 1, wherein said blank matrix or said impregnated matrix is divided into discrete and independent regions.

14. A method for testing at least one chemical entity for the ability of said chemical entity to enhance or inhibit a biological process, said method comprising the steps of:
(a) providing a blank matrix having at least two major surfaces, said at least two major surfaces capable of receiving assay components and chemical entities;
(b) applying at least one assay component required for a biological process to at least one of said at least two major surfaces of said blank matrix, whereby an impregnated matrix is formed;
(c) applying to at least one of said at least two major surfaces of said impregnated matrix at least one chemical entity; and
(d) evaluating the ability of said at least one chemical entity to enhance or inhibit said biological process involving said at least one assay component.

15. The method of claim 14, wherein step (d) includes the step of detecting a response that indicates whether said at least one chemical entity enhances or inhibits said biological process involving said at least one assay component.

16. The method of claim 15, further including the step of preparing an image of said detected response.

17. The method of claim 14, wherein said at least one assay component is applied to at least one of said at least two major surfaces of said blank matrix by means of a transfer sheet.

18. The method of claim 14, wherein said at least one assay component is applied to at least one of said at least two major surfaces of said blank matrix by means of liquid dispensing apparatus.

19. The method of claim 14, wherein said at least one assay component entity is applied to at least one of said at least two major surfaces of said blank matrix in the form of a solid.

20. The method of claim 14, wherein said at least one chemical entity is applied to at least one of said at least two major surfaces of said impregnated matrix by means of a transfer sheet.

21. The method of claim 14, wherein said at least one chemical entity is applied to at least one of said at least two major surfaces of said impregnated matrix by means of liquid dispensing apparatus.

22. The method of claim 14, wherein said at least one chemical entity is applied to at least one of said at least two major surfaces of said impregnated matrix in the form of a solid.

23. The method of claim 14, wherein at least two different assay components are applied to said blank matrix.

24. The method of claim 14, wherein at least two different chemical entities are applied to said impregnated matrix.

25. The method of claim 14, wherein one or more additional chemical entities or one or more additional assay components can be applied to at least one of said at least two major surfaces of said impregnated matrix.

26. The method of claim 14, wherein said blank matrix or said impregnated matrix is divided into discrete and independent regions.
